(19) United States
(12) Patent Application Publication
(10) Pub. No.: US 2024/0173197 A1
(43) Pub. Date: May 30, 2024

(54) DEVICE FOR PHYSIOTHERAPEUTIC TREATMENT OF THE HUMAN BODY, CONSISTING OF TWO DISCS WITH AN INTERPOSED KINESIOLOGIC TAPE, AND METHOD FOR PHYSIOTHERAPEUTIC TREATMENT OF THE HUMAN BODY

(71) Applicant: Jan Niclas WINTER, Rodgau (DE)

(72) Inventor: Jan Niclas WINTER, Rodgau (DE)

(21) Appl. No.: 18/285,368

(22) PCT Filed: Feb. 25, 2022
(86) PCT No.: PCT/DE2022/100159

§ 371 Date: Oct. 2, 2023

(30) Foreign Application Priority Data
Apr. 3, 2021 (DE) 202021001254.0

(51) Int. Cl. A61H 7/00

(52) U.S. Cl. CPC A61H 7/001; A61H 2201/165; A61H 2201/1695

(57) ABSTRACT

The invention relates to a device for physiotherapeutic treatment of the human body, wherein the device consists of two round discs that can be assembled via an anchoring pin in the first round disc and a cavity in the second round disc, and the second round disc has a conical protrusion on the bottom side, which is placed on the human skin during treatment and thereby applies pressure to the skin in a pointwise manner via the second round disc when pressure is applied to the first round disc, thereby reducing tension and stimulating blood circulation. The invention also relates to a method for physiotherapeutic treatment of the human body, in which mechanical pressure is applied to the first round disc that is transferred to the skin in a pointwise manner via the second round disc.

The invention relates to a device for physiotherapeutic treatment of the human body, wherein the device consists of two round discs that can be assembled through an anchoring pin in the first round disc and a cavity in the second round disc, and the second round disc has a conical protrusion on the bottom side, which is placed on the human skin during treatment and thereby applies pointwise pressure to the skin via the second round disc when pressure is applied to the first round disc, thereby reducing tension and stimulating blood circulation. The invention also relates to a method for physiotherapeutic treatment of the human body, in which mechanical pressure is applied to the first round disc and transferred to the skin in a pointwise manner via the second round disc.

In many procedures of the treatment of the human body through physiotherapy, mechanical pressure is applied to the human skin and the underlying body. The use of mechanical pressure increases blood circulation in the area where the pressure is applied, relieves tension within the muscles, and supports lymphatic drainage. The simplest example of the application of mechanical pressure to human skin is massage. In massage, mechanical pressure is applied to the skin and the underlying body by the human hand to relieve muscle tension and stimulate blood circulation. The pressure pattern is usually quite simple and depends on the strength and skill of the masseur. However, in many areas of the human body, the problematic locations are often only located in a small and narrowly bounded region. For example, it is possible that pain and circulatory disorders only develop at certain narrowly bounded areas of the human body. In this case, a wide-area treatment is often not desired, because the treatment at the actual painful locations is too weak and the pain-free areas of the body are exposed to unwanted mechanical pressure.

An example of problematic locations in the body that are situated in a small and narrowly bounded region are the so-called trigger points. Trigger points are small tensions within the musculature, which are associated with reduced local blood circulation and can cause pain when active. These trigger points can be treated using various manual techniques in physiotherapy. These are usually pressure treatments. The pressure treatment must then be performed exactly at the trigger point so that the tensions within the musculature are reduced and blood circulation is also stimulated in a pointwise manner. Also, muscle tensions can often expand into so-called myogeloses, where the affected muscles harden. These myogeloses are usually very painful. Myogelosis and muscle tension should only be treated in the affected area.

A massage performed by hand is often insufficient to achieve the desired results. On one hand, the application of mechanical pressure with the hand depends on the physiognomy and muscle strength distribution of the masseur. On the other hand, a narrowly defined application of mechanical pressure, for example in the treatment of trigger points, is often not possible due to the size of the human hand. Therefore, in the related art, there are many aids to enable a more targeted application of mechanical pressure on the human skin than is possible with the human hand.

Document EP0199872A1 describes a magnetic therapeutic instrument, which includes a permanent magnet, metallic tapes that are each connected at their rear end to the back of the permanent magnet, an adhesive tape that is placed on the permanent magnet, and a removable sheet of paper that is placed on the adhesive tape on the side opposite the permanent magnet, and a germanium layer that has been applied on the side of the permanent magnet opposite the adhesive tape and is 0.05 to 30 μm thick, wherein the permanent magnet is oriented with the N-polarity towards the germanium layer and has a magnetic field strength of 750 to 850 Oersted, and the germanium layer is aligned such that the C-axis is in line with the magnetic axis. For therapeutic treatment of the human body, the magnetic therapeutic instrument is applied with the germanium layer to the skin of the human body, whereby an electric current flows through the human body while the germanium layer wears off, thereby reducing pain and stiffness in the limbs and particularly in the shoulders.

Document CN213941156U describes a patch for placing onto the face of a patient with sleep disorders, wherein the patch has a mushroom shape and the wide, horizontal upper part of the patch is intended for placing onto the forehead and the narrow lower part is intended for placing onto the nasal area, and perpendicular to the patch in the transition area from the upper wide part to the lower narrow part a pressure rod is provided, which serves to press the patch onto the so-called Yintang point, whereby after overnight use the ability to concentrate is strengthened and insomnia is eliminated.

This related art describes devices that are placed onto the patient's skin instead of the human hand to reduce pain or other physical ailments. These devices apply electric current to the skin or apply a pointwise mechanical pressure to a precisely predetermined location on the skin Pointwise pressure on the skin, especially on a precisely predetermined location regardless of the location of the occurring pain, is often not desired in a physiotherapeutic treatment. The pain and ailments that are treatable by physiotherapy are usually not distributed in a precise pointwise manner but extend over a small and narrowly bounded region of the skin and the underlying body. Moreover, these can occur at any location on the human body.

Therefore, it would be desirable to have a device that absorbs the mechanical pressure of the hand during physiotherapy and distributes it over a specified area of the skin and the underlying human body. This device should not distribute the mechanical pressure of the hand pointwise onto the human skin but over a narrowly bounded region of the skin and the underlying body. A certain topology of the device would be desirable, which would mechanically stimulate the skin and the underlying body due to this topology. This device should also apply the mechanical pressure as much as possible to a precisely defined narrowly bounded region of the skin to combat the pain present there. This device should also be usable at any location on the body.

A device that absorbs the mechanical pressure from the hand of the masseur and transfers it to any location on the patient's skin is not present in the related art. Therefore, an objective is to provide a device for transferring mechanical pressure from the hand of a masseur to the human skin, by which a narrowly bounded region of human skin is treated and which mechanically stimulates the treated region of the skin and the underlying body due to its topology.

The present invention addresses this objective by providing a device, which is assembled from a first round disc with a planar bottom side and an outwardly bulging top side and a second round disc with a planar top side and an outwardly bulging bottom side.

The top side of the first round disc has a slope, the bulge of which continuously decreases towards the disc center. This results in an overall hemispherical bulge of the top side. The hemispherical bulge is usually flattened to allow good contact with the masseur's hand. The hemispherical bulge of the top side of the first round disc can also be shaped for receiving the masseur's or user's hand. By contrast, the bottom side of the second round disc has a slope, the bulge of which first increases and then decreases towards the disc center. This results in an overall conical bulge of the bottom side. The conical bulge of the bottom side of the second round disc provides a topology of the device that allows good mechanical stimulation of the skin and the underlying body. The limited extent of the device in turn allows good treatment of the narrowly bounded region where the pain and ailments occur. The first and second discs are round, but can also be approximately round in a way that they still fulfill their purpose of a treatment area uniform in size.

The first round disc has an anchoring pin, which has a thicker portion at the lower end and can be locked into a designated cavity protruding into the second round disc. This lock can be released to allow disassembly and storage of the device. The first round disc and the second round disc can be assembled in this way. A physiotherapeutic treatment of the human body within the scope of the present invention only takes place in the assembled state of the device, when the first round disc and the second round disc are assembled and the anchoring pin is locked.

Between the first round disc and the second round disc, there is a kinesiologic tape. This is used to secure the device to the skin during treatment. This allows for precise treatment of the region where pain and ailments occur.

In particular, what is claimed is a device for physiotherapeutic treatment of the human body, comprising
a first round disc with a bottom side and an outwardly bulging top side, wherein the slope of the bulge of the top side decreases continuously towards the disc center, and
a second round disc with a top side and an outwardly bulging bottom side, wherein the slope of the bulge of the bottom side first increases and then decreases towards the disc center,
   and characterized in that
the first round disc has an anchoring pin protruding from the bottom side towards the bottom side, which has a thicker portion, and
the second round disc has a cavity protruding into the second round disc towards the top side, which widens inside the second round disc and which is able to be stretched at the opening of the cavity and has a smaller diameter there than the interior of the cavity, whereby the anchoring pin of the first round disc with the thicker portion is able to be locked in the cavity of the second round disc, and
a kinesiologic tape, which has an opening and can be slipped onto the anchoring pin.

In one embodiment of the invention, the bottom side of the first round disc and the top side of the second round disc are planar. Thereby, the bottom side of the first round disc and the top side of the second round disc interlock well with an interposing kinesiologie tape. It is also possible to form the bottom side of the first round disc and the top side of the second round disc to be non-planar. However, they must then be formed in such a way that they interlock with an interposed kinesiologic tape.

The anchoring pin preferably has the thicker portion at the lower end and the cavity has the widening for receiving the thicker portion at the inner end. This ensures optimal hold of the anchoring pin in the cavity after locking and that the anchoring pin is easily detachable again.

In another embodiment of the invention, the first round disc has a diameter of 10 to 50 mm. This size allows for good contact with the masseur's hand.

In another embodiment of the invention, the second round disc also has a diameter of 10 to 50 mm. This size allows for the treatment of a narrowly bounded region on the skin and the underlying body.

In another embodiment of the invention, the second round disc has a height of 5 to 15 mm. This height allows for a good processing depth of the human skin, such that underlying touch nerves are stimulated, blood circulation of the body parts under the skin is stimulated, tension of the muscles is normalized, and lymphatic drainage is supported.

Typically, the device is present in an embodiment where a kinesiologic tape is slipped onto the anchoring pin and the first round disc is locked into the cavity of the second round disc through the anchoring pin, thereby forming an assembled device with the first round disc, the second round disc, and the interposed kinesiologic tape. As a result, the device is assembled and usable for physiotherapeutic treatment.

Kinesiologic tapes are known in the related art. An example of a kinesiologie tape is given in document EP2524683B1. In the present invention, the anchoring pin is slipped onto the kinesiologic tape. For the slipping onto the anchoring pin, the kinesiologic tape is punched, perforated, or cut, and the anchoring pin is pulled through the resulting punching, perforation, or cut. Subsequently, the anchoring pin is locked into the designated cavity of the second round disc. In a preferred embodiment, the kinesiologic tape for slipping onto the anchoring pin is punched, perforated, or cut in the geometric center. In a preferred embodiment, the punching is a cross-shaped punching in the geometric center of the kinesiologic tape.

Also claimed are the individual components of the device. A first round disc is claimed, having a planar bottom side and an outwardly bulging top side, wherein the slope of the bulge of the top side continuously decreases towards the disc center, and which is characterized in that it has an anchoring pin with a thicker portion on the bottom side. This can be used for assembly to the device according to the invention.

A second round disc is also claimed, having a planar top side and an outwardly bulging bottom side, wherein the slope of the bulge of the bottom side first increases and then decreases towards the disc center, and which is characterized in that it has a cavity protruding into the second round disc towards the planar top side, which widens in the second round disc and which is able to be stretched at the opening of the cavity and has a smaller diameter there than the interior of the cavity. This can be used for assembly to the device according to the invention.

A kinesiologic tape is also claimed, which is characterized in that it has a punching in the geometric center. This can be used for assembly to the device according to the invention.

The aforementioned device can, preferably in assembled form with the interposing kinesiologic tape, be used for physiotherapeutic treatment. Therefore, the use of the device in a method for physiotherapeutic treatment of the human body is also claimed. Physiotherapeutic treatments are understood to include all forms of treatment of the human body that intend to heal diseases and ailments through non-invasive steps. This also includes sports orthopedic treatments. Also claimed is a method for physiotherapeutic treatment of the human body, in which the above-described assembled device or one of the above-described embodiments of the device is placed with the bottom side of the second round disc on the skin of the human body and acts on it by applying mechanical pressure from the outside via the first round disc. The use is usually performed over a short treatment period.

In one embodiment of the method for physiotherapeutic treatment of the human body, the placing of the above-described assembled device or one of the above-described embodiments and the physiotherapeutic treatment are performed on a so-called trigger point. It is also possible to perform a physiotherapeutic treatment by placing the above-described assembled device or one of the above-described embodiments onto a myogelosis or a tensed muscle.

In another embodiment of the method for physiotherapeutic treatment of the human body, the treatment is performed over a period of 1 to 5 days. As a result, the treated trigger point, the myogelosis, or the muscle tension is deactivated, the pain is alleviated, and normal tension in the muscle is achieved.

The invention has the advantage of providing a device for physiotherapeutic treatment of the human body, which exerts mechanical pressure on a precisely defined narrowly bounded region of the skin to combat the pain present there and mechanically stimulates a precisely defined narrowly bounded region of the skin and the underlying body due to its topology.

The invention is further explained with the help of seven drawings, wherein these drawings only represent embodiments and the invention is not limited to these embodiments.

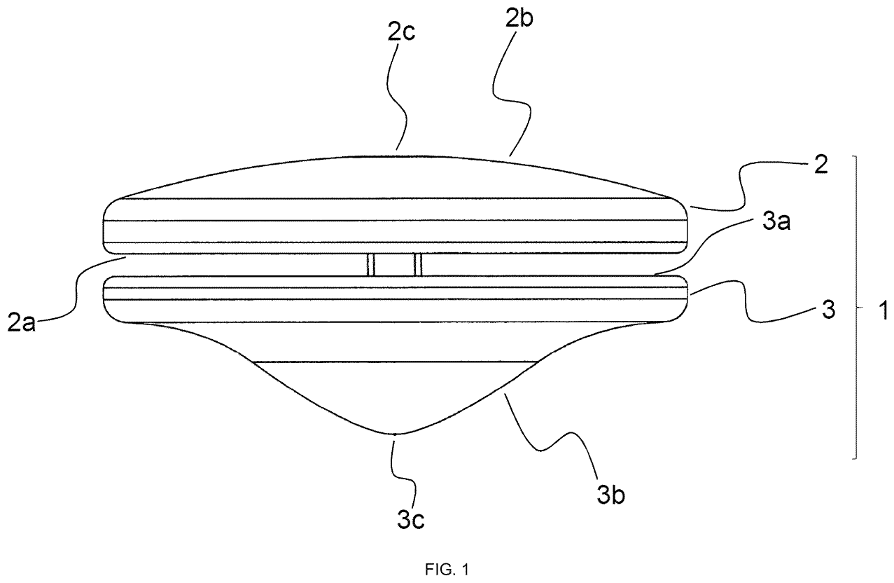

The drawing FIG. 1 shows the device according to the invention with a first round disc and a second round disc in a side view.

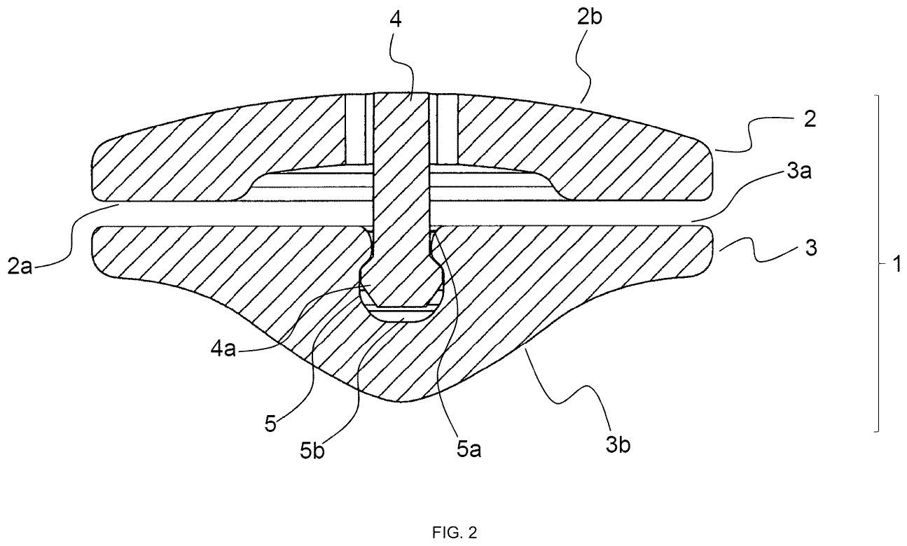

The drawing FIG. 2 shows a cross-sectional drawing of the device according to the invention.

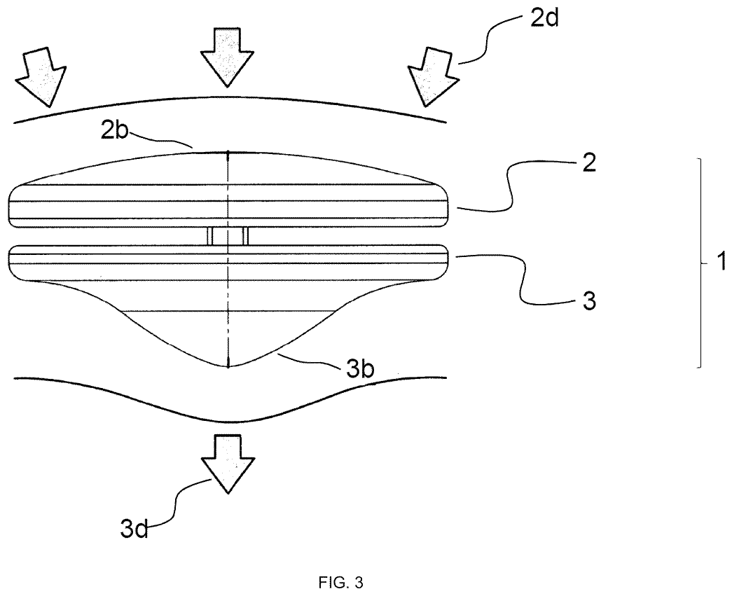

The drawing FIG. 3 shows the device according to the invention during a physiotherapeutic treatment in a side view.

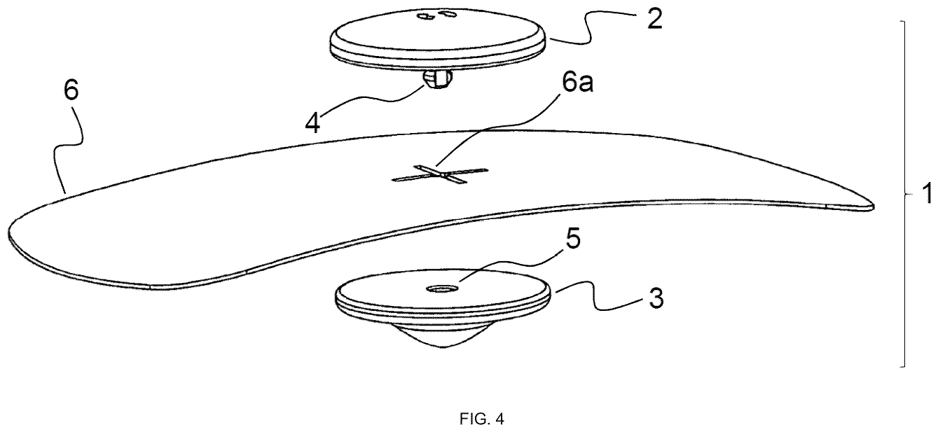

The drawing FIG. 4 shows the device according to the invention with the first round disc and the second round disc in a separated state in an oblique side view from above.

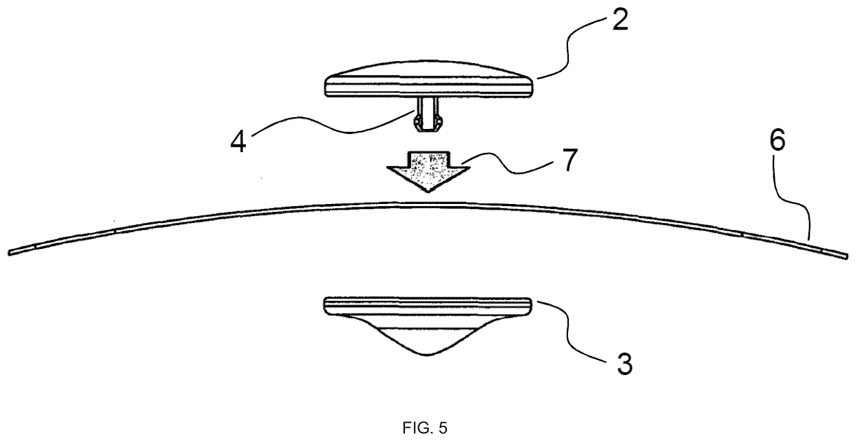

The drawing FIG. 5 shows the device according to the invention in a separated state with a kinesiologic tape between the first and the second round discs in a side view.

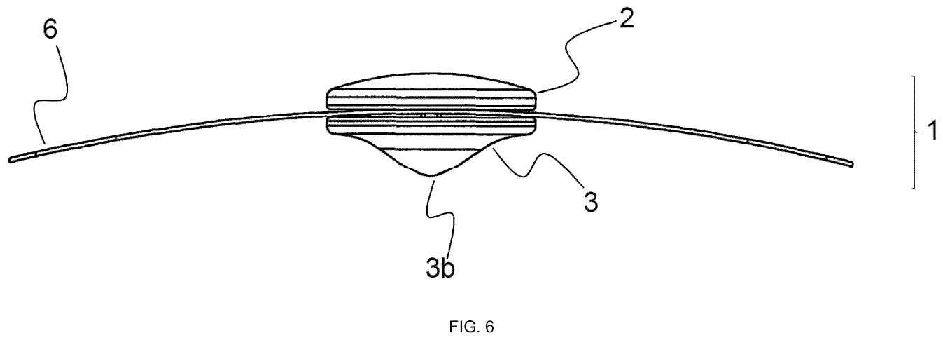

The drawing FIG. 6 shows the device according to the invention in an assembled state with the kinesiologic tape in a side view.

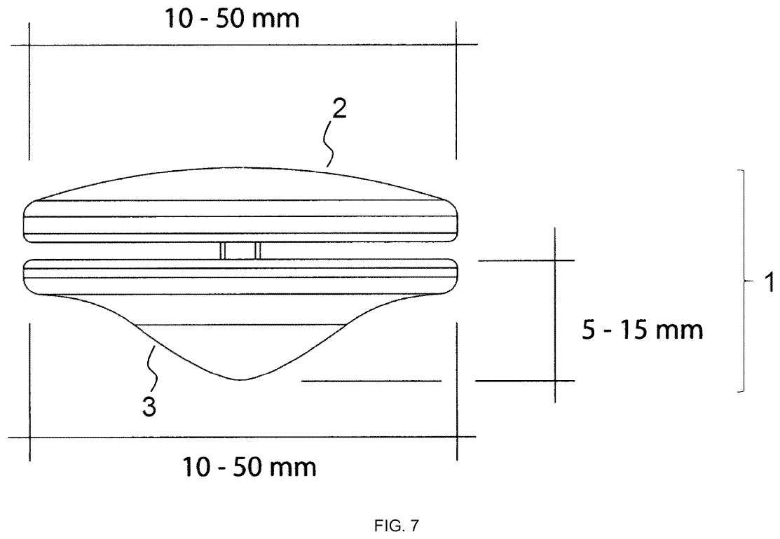

The drawing FIG. 7 shows the device according to the invention in an assembled state in a side view with the corresponding dimensions.

The drawing FIG. 1 shows the device (1) according to the invention with a first round disc (2) and a second round disc (3) in a side view. The first round disc (2) has a planar bottom side (2a) and an outwardly bulging top side (2b), wherein the slope of the bulge of the top side (2b) continuously decreases towards the disc center (2c). The second round disc has a planar top side (3a) and an outwardly bulging bottom side (3b), where the slope of the bulge of the bottom side (3b) first increases and then decreases towards the disc center (3c). As a result, the second round disc (3) has a conical bottom side (3b).

The drawing FIG. 2 shows a cross-sectional drawing of the device (1) according to the invention with the first round disc (2) and the second round disc (3). At the first round disc (2), the anchoring pin (4) can be seen, which has a thicker portion (4a) at the lower end and which can be detachably locked into a protruding cavity (5) provided for this purpose in the second round disc (3). The cavity (5) can be stretched at the opening (5a), has a smaller diameter there than the interior of the cavity (5), and widens towards the inner end (5b). As a result, the anchoring pin (4) of the first round disc (2) with the thicker portion (4a) at the inner end can be locked into the wider portion (5b) of the cavity (5) of the second round disc (3).

The drawing FIG. 3 shows the device (1) according to the invention with the first round disc (2) and the second round disc (3) during a physiotherapeutic treatment in a side view. Mechanical pressure (2d) is applied to the bulging top side (2b) of the first round disc (2) by hand in a way that the conical bottom side (3b) of the second round disc (3) exerts mechanical pressure (3d) on the underlying human skin. Due to the conical topology of the bottom side (3b) of the second round disc (3), the skin and the underlying body are mechanically stimulated.

The drawing FIG. 4 shows the device (1) according to the invention with the first round disc (2) and the second round disc (3) in a separated state in an oblique side view from above. A kinesiologic tape (6) is arranged between the first round disc (2) and the second round disc (3). Also visible are the anchoring pin (4) and the cavity (5) protruding into the second round disc (3). The kinesiologic tape (6) has a cross-shaped punching (6a) in the geometric center, through which the anchoring pin (4) can be pulled and the first round disc (2) and the second round disc (3) with the interposed kinesiologic tape (6) can be assembled into the device (1) according to the invention.

The drawing FIG. 5 shows the device (1) according to the invention with the first round disc (2) and the second round disc (3) in a separated state with the kinesiologic tape (6) between the first round disc (2) and the second round disc (3) in a side view. The first round disc (2) is inserted (7) into the second round disc (3) via the anchoring pin (4), wherein the kinesiologic tape (6) is inserted between the first round disc (2) and the second round disc (3) and the anchoring pin (4) is guided through in the kinesiologic tape (6) via the punching (6a).

The drawing FIG. 6 shows the device (1) according to the invention with the first round disc (2) and the second round disc (3) in an assembled state with the kinesiologic tape (6) between the first round disc (2) and the second round disc (3) in a side view. For use in physiotherapeutic treatment, the conical bottom side (3b) is placed on the human skin to be treated and is attached to the human skin via the kinesiologic tape (6).

The drawing FIG. 7 shows the device (1) according to the invention with the first round disc (2) and the second round disc (3) in an assembled state in a side view with the corresponding dimensions. The first round disc (2) and the second round disc (3) each have a diameter of 10 to 50 mm. The second round disc (3) has a height of 5 to 15 mm.

### LIST OF REFERENCE NUMERALS

1 Device
2 First round disc
2a Bottom side of the first round disc
2b Top side of the first round disc
2c Disc center of the first round disc
2d Mechanical pressure of the hand
3 Second round disc
3a Top side of the second round disc
3b Bottom side of the second round disc
3c Disc center of the second round disc
3d Mechanical pressure on the human skin
4 Anchoring pin
4a Thicker portion
5 Cavity
5a Opening
5b Wider portion
6 Kinesiologic tape
6a Punching in the shape of a cross
7 Insertion process

1. A device (1) for physiotherapeutic treatment of the human body, comprising
a first round disc (2) with a bottom side (2a) and an outwardly bulging top side (2b), wherein the slope of the bulge of the top side (2b) decreases continuously towards the disc center (2c), and
a second round disc (3) with a top side (3a) and an outwardly bulging bottom side (3b), wherein the slope of the bulge of the bottom side (3b) first increases and then decreases towards the disc center (3c),
characterized in that
the first round disc (2) has an anchoring pin (4) protruding from the bottom side (2a) towards the bottom side (2a), which has a thicker portion (4a), and
the second round disc (3) has a cavity (5) protruding into the second round disc (3) towards the top side (3a), which widens inside the second round disc (3) and which is able to be stretched at the opening of the cavity (5) and has a smaller diameter there than the interior of the cavity (5), whereby the anchoring pin (4) of the first round disc (2) with the thicker portion (4a) is able to be locked in the cavity (5) of the second round disc (3), and
a kinesiologic tape (6), which has an opening (6a) and can be slipped onto the anchoring pin (4).

2. The device (1) according to claim 1, characterized in that the bottom side (2a) of the first round disc (3) and the top side of the second round disc are planar.

3. The device (1) according to any of claim 1 or 2, characterized in that the anchoring pin (4) has the thicker portion (4a) at the lower end and the cavity (5) has a wider portion (Sb) at the inner end for receiving the thicker portion (4a).

4. The device (1) according to any of claims 1 to 3, characterized in that the first round disc (2) has a diameter of 10 to 50 mm.

5. The device (1) according to any of claims 1 to 4, characterized in that the second round disc (3) has a diameter of 10 to 50 mm.

6. The device (1) according to any of claims 1 to 5, characterized in that the second round disc (3) has a height of 5 to 15 mm.

7. The device (1) according to any of claims 1 to 6, characterized in that the kinesiologic tape (6) is slipped onto the anchoring pin (4) and the first round disc (2) is locked into the cavity (5) of the second round disc (3) through the anchoring pin (4), whereby the first round disc (2) and the second round disc (3) with the interposed kinesiologic tape (6) form an assembled device (1).

8. A first round disc (2) with a bottom side (2a) and an outwardly bulging top side (2b), wherein the slope of the bulge of the top side decreases continuously towards the disc center (2c), characterized in that it has an anchoring pin (4) with a thicker portion (4a) on the bottom side (2a).

9. A second round disc (3) with a top side (3a) and an outwardly bulging bottom side (3b), wherein the slope of the bulge of the bottom side first increases and then decreases towards the disc center (3c), characterized in that it has a cavity (5) protruding into the round disc (3) towards the top side (3a) which widens in the second round disc (3) and which is able to be stretched at the opening of the cavity (5) and has a smaller diameter there than the interior of the cavity (5).

10. A kinesiologic tape (6), characterized in that it has a punching (6a) in the geometric center.

11. A method for physiotherapeutic treatment of the human body, characterized in that an assembled device (1) according to any of the claims 1 to 7 is placed with the bottom side (3b) of the second round disc (3) onto the skin of the human body and acts on it (3d) through the application of pressure (2d) from the outside via the first round disc (2).

12. The method according to claim 11, characterized in that the placing of the device (1) is performed on a so-called trigger point.

13. The method according to claim 11, characterized in that the placing of the device (1) is performed on a myogelosis or a tensed muscle.

14. The method according to any of claim 12 or 13, characterized in that the treatment is performed over a period of 1 to 5 days.
